(19) United States
(12) Patent Application Publication
(10) Pub. No.: US 2002/0081342 A1
(43) Pub. Date: Jun. 27, 2002

(54) Cough suppressant composition

(76) Inventor: Mostafa K. Behzadpour, San Rafael, CA (US)

Correspondence Address: Kaardal & Associates, PC, Attn: Ivar M. Kaardal, Suite 250, 3500 South First Ave. Circle, Sioux Falls, SD, 57105-5802, US

(21) Appl. No.: 09/747,889

(22) Filed: Dec. 26, 2000

(51) Int. Cl. A61K 35/78

(52) U.S. Cl. 424/734

(57) ABSTRACT

A cough suppressant composition for successfully treating coughs. The cough suppressant composition includes black pepper and at least one flavorant diluted in an aqueous solution.

### BACKGROUND OF THE INVENTION

1. Field of the Invention

The present invention relates to a cough suppressant and more particularly pertains to a new cough suppressant composition for successfully treating coughs.

2. Description of the Prior Art

The use of a cough suppressant is known in the prior art. More specifically, a cough suppressant heretofore devised and utilized are known to consist basically of familiar, expected and obvious structural configurations, notwithstanding the myriad of designs encompassed by the crowded prior art which have been developed for the fulfillment of countless objectives and requirements.

Known prior art includes U.S. Pat. No. 5,376,374; U.S. Pat. No. 6,004,560; U.S. Pat. No. 5,035,882; U.S. Pat. No. 5,897,897; U.S. Pat. No. 5,736,144; and U.S. Pat. No. 4,311,720.

While these devices fulfill their respective, particular objectives and requirements, the aforementioned patents do not disclose a new cough suppressant composition. The inventive device includes black pepper and at least one flavorant diluted in an aqueous solution.

In these respects, the cough suppressant composition according to the present invention substantially departs from the conventional concepts and designs of the prior art, and in so doing provides an apparatus primarily developed for the purpose of successfully treating coughs.

### SUMMARY OF THE INVENTION

In view of the foregoing disadvantages inherent in the known types of cough suppressant now present in the prior art, the present invention provides a new cough suppressant composition construction wherein the same can be utilized for successfully treating coughs.

The general purpose of the present invention, which will be described subsequently in greater detail, is to provide a new cough suppressant composition which has many of the advantages of the cough suppressant mentioned heretofore and many novel features that result in a new cough suppressant composition which is not anticipated, rendered obvious, suggested, or even implied by any of the prior art cough suppressant, either alone or in any combination thereof.

To attain this, the present invention generally comprises black pepper and at least one flavorant diluted in an aqueous solution.

There has thus been outlined, rather broadly, the more important features of the invention in order that the detailed description thereof that follows may be better understood, and in order that the present contribution to the art may be better appreciated. There are additional features of the invention that will be described hereinafter and which will form the subject matter of the claims appended hereto.

In this respect, before explaining at least one embodiment of the invention in detail, it is to be understood that the invention is not limited in its application to the details of construction and to the arrangements of the components set forth in the following description or illustrated in the drawings. The invention is capable of other embodiments and of being practiced and carried out in various ways. Also, it is to be understood that the phraseology and terminology employed herein are for the purpose of description and should not be regarded as limiting.

As such, those skilled in the art will appreciate that the conception, upon which this disclosure is based, may readily be utilized as a basis for the designing of other structures, methods and systems for carrying out the several purposes of the present invention. It is important, therefore, that the claims be regarded as including such equivalent constructions insofar as they do not depart from the spirit and scope of the present invention.

Further, the purpose of the foregoing abstract is to enable the U.S. Patent and Trademark Office and the public generally, and especially the scientists, engineers and practitioners in the art who are not familiar with patent or legal terms or phraseology, to determine quickly from a cursory inspection the nature and essence of the technical disclosure of the application. The abstract is neither intended to define the invention of the application, which is measured by the claims, nor is it intended to be limiting as to the scope of the invention in any way.

It is therefore an object of the present invention to provide a new cough suppressant composition which has many of the advantages of the cough suppressant mentioned heretofore and many novel features that result in a new cough suppressant composition which is not anticipated, rendered obvious, suggested, or even implied by any of the prior art cough suppressant, either alone or in any combination thereof.

It is another object of the present invention to provide a new cough suppressant composition which may be easily and efficiently manufactured and marketed.

It is a further object of the present invention to provide a new cough suppressant composition which is of a durable and reliable construction.

An even further object of the present invention is to provide a new cough suppressant composition which is susceptible of a low cost of manufacture with regard to both materials and labor, and which accordingly is then susceptible of low prices of sale to the consuming public, thereby making such cough suppressant composition economically available to the buying public.

Still yet another object of the present invention is to provide a new cough suppressant composition which provides in the apparatuses and methods of the prior art some of the advantages thereof, while simultaneously overcoming some of the disadvantages normally associated therewith.

Still another object of the present invention is to provide a new cough suppressant composition for successfully treating coughs.

Yet another object of the present invention is to provide a new cough suppressant composition which includes black pepper and at least one flavorant diluted in an aqueous solution.

Still yet another object of the present invention is to provide a new cough suppressant composition that is quite effective and fast in the suppression of coughs.

Even still another object of the present invention is to provide a new cough suppressant composition that eliminates the potential for adverse reactions from medications.

These together with other objects of the invention, along with the various features of novelty which characterize the invention, are pointed out with particularity in the claims annexed to and forming a part of this disclosure. For a better understanding of the invention, its operating advantages and the specific objects attained by its uses, reference should be made to the accompanying drawings and descriptive matter in which there are illustrated preferred embodiments of the invention.

### BRIEF DESCRIPTION OF THE DRAWINGS

The invention will be better understood and objects other than those set forth above will become apparent when consideration is given to the following detailed description thereof. Such description makes reference to the annexed drawings wherein:

FIG. 1 is a schematic diagram of a new cough suppressant composition according to the present invention.

### DESCRIPTION OF THE PREFERRED EMBODIMENT

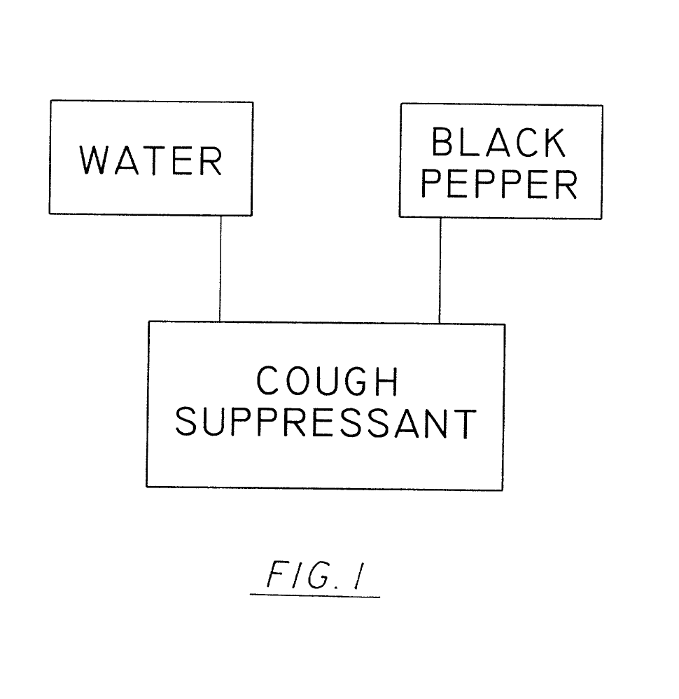

With reference now to the drawings, and in particular to FIG. 1 thereof, a new cough suppressant composition embodying the principles and concepts of the present invention and generally designated by the reference numeral 10 will be described.

As best illustrated in FIG. 1, the cough suppressant composition 10 generally comprises black pepper and at least one flavorant diluted in an aqueous solution. The aqueous solution of which comprises black pepper and at least one flavorant includes at least 85% by volume of water with the remaining balance being that of black pepper and at least one flavorant. The aqueous solution further includes approximately one part of black pepper and from ⅓ to ⅔ parts of the at least one flavorant to 16 parts of water. The aqueous solution also includes approximately one tablespoon of black pepper; from one teaspoon to two teaspoons of at least one flavorant; and approximately 8 fluid ounces of water. The black pepper is essentially black pepper corn with the at least one flavorant being sugar and lemon juice.

In use, the aqueous solution including black pepper, sugar and lemon juice is brought to a boil and is then allowed to cool and stored for later use. When needed, the cooled solution is consumed by the user by drinking the solution without any need to worry about how much to take.

As to a further discussion of the manner of usage and operation of the present invention, the same should be apparent from the above description. Accordingly, no further discussion relating to the manner of usage and operation will be provided.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A cough suppressant composition comprising:
black pepper and at least one flavorant diluted in an aqueous solution.

2. A cough suppressant composition as described in claim 1, wherein said aqueous solution comprising said black pepper and said at least one flavorant includes at least 85% by volume of water with a remaining balance being that of said black pepper and said at least one flavorant.

3. A cough suppressant composition as described in claim 2, wherein said aqueous solution further includes approximately one part of said black pepper and from ⅓ to ⅔ parts of said at least one flavorant to 16 parts of said water.

4. A cough suppressant composition as described in claim 3, wherein said aqueous solution also includes approximately one tablespoon of said black pepper; from one teaspoon to two teaspoons of said at least one flavorant; and approximately 8 fluid ounces of said water.

5. A cough suppressant composition as described in claim 4, wherein said black pepper is essentially black pepper corn.

6. A cough suppressant composition as described in claim 5, wherein said at least one flavorant includes sugar and lemon juice.

7. A cough suppressant composition comprising:
black pepper and at least one flavorant diluted in an aqueous solution, said aqueous solution of which comprises said black pepper and said at least one flavorant including at least 85% by volume of water with a remaining balance being that of said black pepper and said at least one flavorant, said aqueous solution further including approximately one part of said black pepper and from ⅓ to ⅔ parts of said at least one flavorant to 16 parts of said water, said aqueous solution also including approximately one tablespoon of said black pepper; from one teaspoon to two teaspoons of said at least one flavorant; and approximately 8 fluid ounces of said water, said black pepper being essentially black pepper corn, said at least one flavorant including sugar and lemon juice.

8. A method of making a cough suppressant composition includes the steps of:
combining approximately one tablespoon of black pepper with eight ounces of water; one teaspoon of sugar; and one teaspoon of lemon juice in a solution;
heating said solution to boiling;
cooling and storing said solution for use through drinking of said solution.
